(19) United States
(12) Patent Application Publication
(10) Pub. No.: US 2016/0023402 A1
(43) Pub. Date: Jan. 28, 2016

(54) PLASTIC WELDING SYSTEM AND METHOD OF MAKING MEDICAL DEVICE

(71) Applicant: Cook Medical Technologies LLC, Bloomington, IN (US)

(72) Inventor: David Burton, Bloomington, IN (US)

(21) Appl. No.: 14/728,285

(22) Filed: Jun. 2, 2015

Related U.S. Application Data
(60) Provisional application No. 62/029,626, filed on Jul. 28, 2014.

(51) Int. Cl. B29C 65/78; A61M 25/10; B29C 65/68; B29C 65/20; B29C 65/00

(52) U.S. Cl. CPC B29C 65/7844; B29C 65/20; B29C 66/63; B29C 65/68; A61M 25/1029; A61M 25/1034; B29L 2031/7543

(57) ABSTRACT

Making a medical device includes narrowing an aperture of an iris formed by movable contactors in a welding system so as to contact heated surfaces of the movable contactors with the assembly. Heat conducted from the heated surfaces into the assembly softens materials forming at least one of a first and a second components so as to form a plastic weld bonding the first and second components together.

### TECHNICAL FIELD

The present disclosure relates generally to medical device manufacturing and construction, and relates more particularly to forming a plastic weld between first and second components via heated surfaces of movable contactors in an adjustable iris.

### BACKGROUND

Plastic welding is employed throughout the medical device industry to bond together different components formed of similar or different materials. Balloon catheters, infusion bags, and a great many different interventional devices, diagnostic tools, and still other contrivances require or benefit from plastic welding of components. In the case of tube joining and the like current welding machines typically use fixed dies that must be specialized to size. Specialized dies tend to be challenging and expensive to manufacture, require significant time in-process, and have various shortcomings respecting finished product quality.

One fusion joining device for plastic tubes is shown in U.S. Pat. No. 7,015,421 to Nakagawa. The Nakagawa device employs a pair of clampers supported so as to be pivoted between closed positions sandwiching a joint from both sides, to open positions. While Nakagawa may be suitable for its intended purpose, there is always room for improvement.

### SUMMARY OF THE DISCLOSURE

In one aspect, a method of making a medical device includes advancing an assembly of a first component and a second component through an aperture in an iris formed by a plurality of movable contactors in a welding system, and adjusting the iris to narrow the aperture such that heated surfaces of the plurality of movable contactors contact the assembly. The method further includes conducting heat from the heated surfaces into the assembly via the contact so as to soften material forming at least one of the first and second components at an interface therebetween. The method still further includes forming a plastic weld via the softening and subsequent hardening of the material at the interface so as to bond together the first and second components.

In another aspect, a plastic welding system includes a housing, and a plurality of movable contactors mounted to the housing and forming an iris. The iris defines a center axis and is configured to receive therethrough an assembly of a first component and a second component to be bonded together via plastic welding. The plurality of movable contactors each further include a surface oriented toward the center axis, such that the surfaces define an aperture of the iris adjustable in size via moving the plurality of movable contactors. The system further includes a heating mechanism coupled to the plurality of movable contactors so as to heat the surfaces for conducting heat into the assembly. The system still further includes at least one actuator operably coupled to the plurality of movable contactors so as to narrow the aperture to contact the assembly via the surfaces and thereby form a plastic weld bonding together the first and second components.

### BRIEF DESCRIPTION OF THE DRAWINGS

FIG. 1 is a perspective view of a plastic welding system, according to one embodiment;

FIG. 2 is a side diagrammatic view, partially in cutaway, of a part of the system of FIG. 1;

FIG. 3 is a side diagrammatic view, partially in cutaway, of a plastic welding system according to another embodiment;

FIG. 4 is a sectioned diagrammatic view through a portion of a plastic welding system and an assembly of components to be plastic welded, and including a detailed enlargement, according to one embodiment;

FIG. 5 is another sectioned diagrammatic view through a portion of a plastic welding system and a welded assembly of components at another stage of processing, according to one embodiment, and also including a detailed enlargement; and

FIG. 6 is a side view of a medical device according to one embodiment.

### DETAILED DESCRIPTION

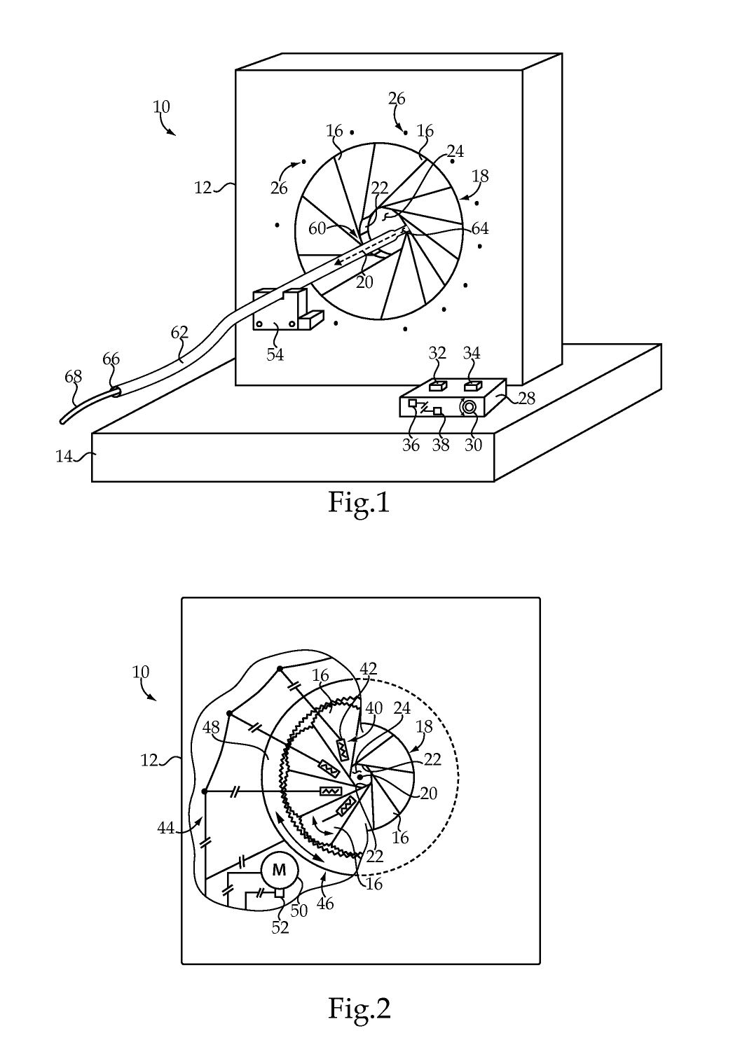

Referring to FIG. 1, there is shown a plastic welding system 10 according to one embodiment. System 10 includes a housing 12, supported upon a base 14. A plurality of movable contactors 16 are mounted to housing 12 and form an iris 18. Iris 18 defines a center axis 20 and is configured to receive therethrough an assembly 60 of a first component 62 and a second component 64 to be bonded together via plastic welding. In FIG. 1, iris 18 is shown as it might appear part way between a fully or maximally closed position, and a fully or maximally open position. Movable contactors 16 each further include a surface 22 oriented toward center axis 20, such that surfaces 22 define an aperture 24 of iris 18 adjustable in size via moving contactors 16. In the illustrated embodiment, contactors 16 are distributed radially symmetrically about center axis 20 and are greater than three in number. Embodiments are contemplated where contactors 16 are about eight to about twelve in number, but potentially greater or lesser than this range. As will be further apparent from the following description, the implementation of an iris for forming plastic welds in components to be bonded together offers various advantages over conventional strategies.

Movable contactors 16 may further be synchronously pivotable about a plurality of pivot axes 26 between first positions where aperture 24 is larger and the corresponding surface 22 is further from center axis 20, and second positions where aperture 24 is smaller and the corresponding surface 22 is closer to center axis 20. In the illustrated embodiment each of movable contactors 16 has a wedge shape, and where surfaces 22 are substantially planar. In other embodiments, surfaces 22 might be curved so that iris 24 is closer to perfectly circular. Each of movable contactors 16 may further be in contact with two other ones of movable contactors 16, and slides in contact with the two other ones between the first and second positions. System 10 may also include a support mechanism or the like 54 mounted to housing 12, so as to support assembly 60 for welding. An operator may additionally or alternatively manually position and support assembly 60 for plastic welding of the same.

Referring also now to FIG. 2, there is shown housing 12 partially in cutaway, and illustrating additional features of system 10. System 10 may further include a heating mechanism 40 coupled to movable contactors 16 so as to heat surfaces 22 for conducting heat into assembly 60. At least one actuator 46 is operably coupled to movable contactors 16 so as to narrow aperture 24 to contact assembly 60 via the respective surfaces 22 and thereby form a plastic weld bonding together the first and second components. In a practical implementation strategy, heating mechanism 40 may include a plurality of resistance heaters 42 each coupled with one of movable contactors 60, for instance attached directly to the movable contactors 16. Electrical lines 44 may connect resistance heaters 42 with a power supply (not shown) in a practical implementation strategy. The at least one actuator 46 may include one electrical actuator 50, such as a motor, coupled to a gear ring 48 that rotates around center axis 20, and is in mesh with gear teeth on each of movable contactors 16. In FIG. 2, a curved arrow on gear ring 48 illustrates approximate directions of rotation and counter rotation of gear ring 48 in response to operation of motor 50 to pivot contactors 16 about their pivot axes 26 to adjust aperture 24 for plastic welding as discussed herein. A curved arrow shown on one of contactors 16 illustrates approximate back and forth pivoting motion of the subject example contactor.

As also illustrated in FIG. 1, system 10 may include an operator input interface 28, including a plurality of control buttons or the like 32 and 34, a control knob 30, and any of a variety of other operator manipulable contrivances. Input interface 28 might be a touch screen, a keyboard, or any other mechanism by which an operator can control opening and closing of iris 18, turn system 10 on or off, adjust a temperature of contactors 16, or control any other parameters of system 10. In a practical implementation strategy, rotation of knob 30 can adjust temperature, button 32 can initiate controllably closing iris 18, and button 34 can initiate controllably opening iris 18.

System 10 may further include a control device 36, such as a data processor, coupled with a computer readable memory 38, and in communication with the one or more actuators 46. Control device 36 may be configured to determine control signals to actuator 46 so as to controllably apply a compressive pressure to assembly 60 during adjustment of the size of aperture 24. In a practical implementation strategy, memory 38 may store computer executable program instructions for controllably applying compressive pressure based upon a pressure selected by an operator and deemed suitable for a particular application, such as plastic welding certain materials. Control device 36 may further be configured to controllably apply condensing pressure via the determined control signals that is uniform at each of first and second positions of movable contactors 16. Also in a practical implementation strategy, motor 50 may include a servo motor, and system 10 may also include a sensor 52 coupled with motor 50 and in communication with control device 36. Control device 36 may utilize inputs from sensor 52 to determine with relatively high precision positions of movable contactors 16, and via controlling motor 50 apply compressive pressure beginning at a point where surfaces 22 first contact assembly 60, and continuing throughout a period of time during which assembly 60 is being plastic welded as described herein. This general strategy of continuous application of compressive pressure as well as heat differs fundamentally from prior strategies, such as the clamping strategies discussed above. In conventional designs, different hole sizes in clamping jaws were used which were considered to be optimal for different sizes of tubes to be joined, but not actively controlled to apply specified pressures, and not even capable of applying specified pressure continuously throughout the course of a plastic welding procedure where the components to be welded shrink and/or material flows.

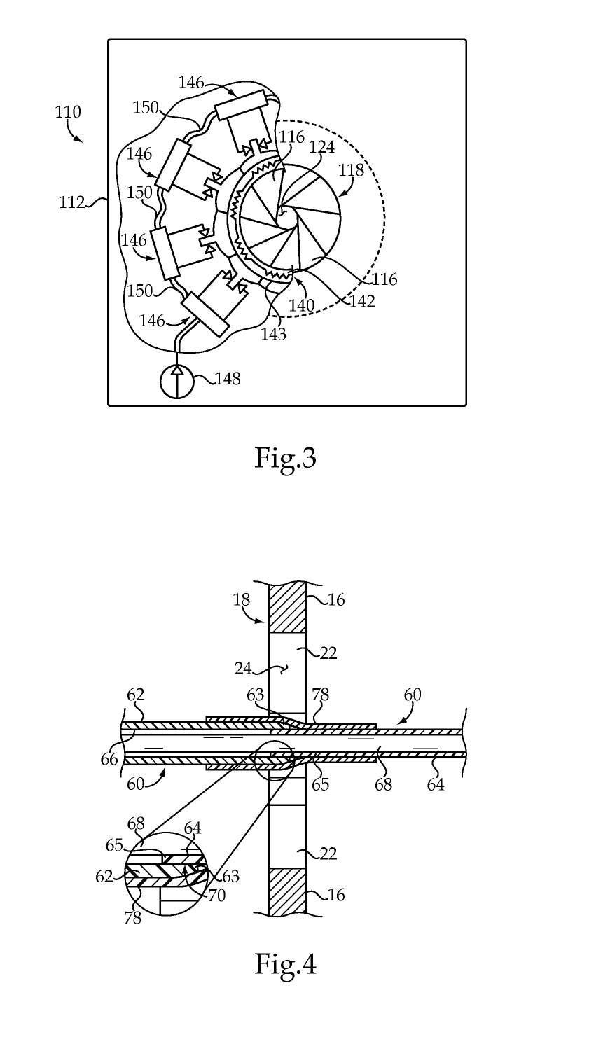

Referring now to FIG. 3, there is shown a plastic welding system 110 according to another embodiment, and also including a housing 112 and a plurality of movable contactors 116 mounted to housing 112 and forming an iris 118. Contactors 116 are movable in housing 112 in a manner analogous to that of contactors 16 in the foregoing embodiment, and define an adjustable size aperture 124 for analogous purposes. System 110 includes a heating mechanism 140 within housing 112 so as to heat surfaces of movable contactors 116 to conduct heat into the assembly to be plastic welded. Heating mechanism 140 also includes one or more resistance heaters 142 mounted to a support ring 143 or the like supported at a fixed position in housing 112, but not directly in contact with movable contactors 116. Contactors 116 might thus be heated via radiating heat from heating mechanism 140 and via convection rather than principally conduction as in the previous embodiments. Another difference between system 110 and system 10 relates to the manner of actuating movable contactors 116 to adjust the size of aperture 124. Rather than an electric motor and gear ring mechanism or the like, actuator 146 includes a pump or compressor 148 coupled with a plurality of individual actuators 146 connected via fluid lines 150 to pump 148. Each of actuators 146 may be operable to pivot movable contactors 116 in alternate directions via applying pressurized fluid to actuation surfaces (not numbered) on contactors 116, as illustrated.

From the foregoing description it will be appreciated that a variety of different heating mechanisms, and a variety of different actuating mechanisms, might be implemented within the context of the present disclosure. Those skilled in the art will also contemplate embodiments where hydraulic actuation of movable contactors is used, or where individual electric motors are coupled with each movable contactor. In still further versions, additional heating and/or cooling apparatus, such as supply lines for heated air to heat the movable contactors or the assembly to be plastic welded, and/or a separate cooling air line to rapidly cool the assembly could be used, depending upon the application.

### INDUSTRIAL APPLICABILITY

Referring to the drawings generally now but in particular to FIG. 4, there is shown a sectioned side view of assembly 60 with iris 18 as it might appear where assembly 60 has been advanced through aperture 24 so as to position assembly 60 to be contacted via surfaces 22 where iris 18 is adjusted to narrow aperture 24. It can be seen from FIG. 4 that each of first and second components 62 and 64 has a tubular shape, and together form a lumen 66. A form 68, in the nature of an elongate metal wire or the like, has been passed through first and second components 62 and 64 to prevent narrowing of lumen 66 during conducting heat from surfaces 22 into assembly 60 via contact therebetween. Form 68 is removable, and will be pulled out of assembly 60 after plastic welding is complete. Also shown in FIG. 4 is a shrink tubing piece 78 that is positioned about components 62 and 64 so as to extend through aperture 24, and to be contacted by surfaces 22.

In the example illustration of assembly 60, a first end 63 of component 62 receives a second end 65 of component 64, such that the coaxial nested ends 63 and 65 are positioned within aperture 24. In the detailed enlargement of FIG. 4, an interface 70 between components 62 and 64 is shown. It will be understood that interface 70 is generally a cylindrical interface as could be expected with coaxial nested tubular components. Where the shape of either of the components 62 or 64 is varied, or the manner in which they overlap one another is varied, the shape of interface 70 could be non-cylindrical. Also, while the extent of overlap is relatively modest, being less than an axial thickness or extent of iris 18, in other embodiments the overlap could be relatively more extensive.

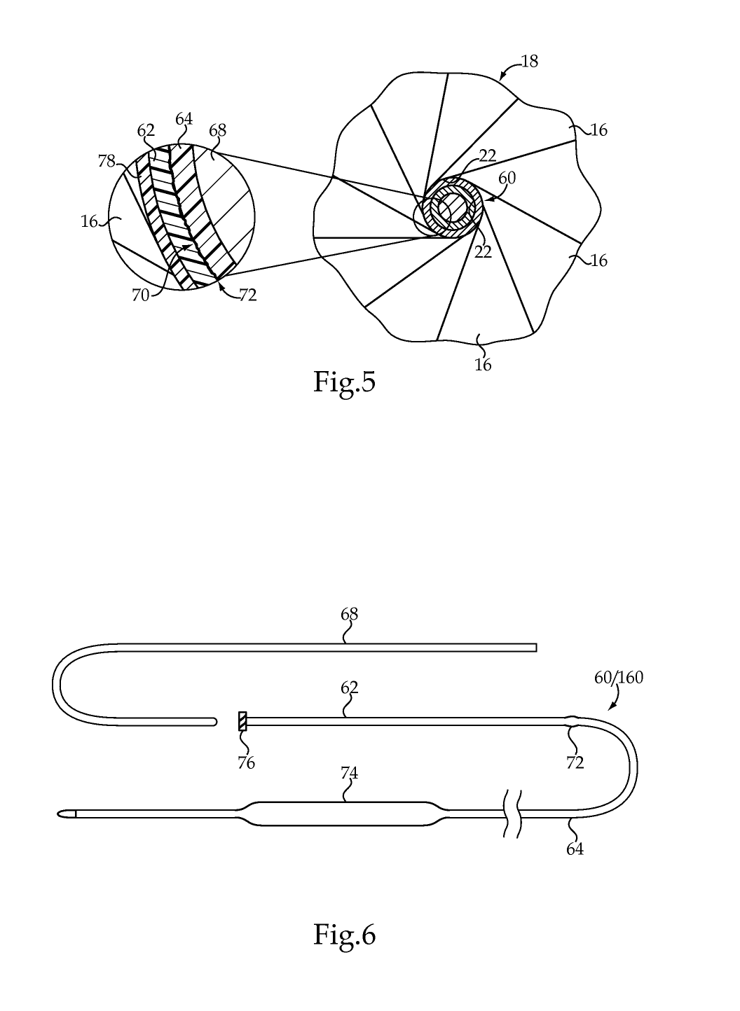

Referring also now to FIG. 5, there is shown an axial view, partially sectioned, through assembly 60 within iris 18, where movable contactors 16 have been adjusted via narrowing aperture 24 such that heated surfaces 22 contact assembly 60. Heat may then be conducted from surfaces 22 into assembly 60 via the contact so as to soften material forming at least one of first component 62 and second component 64 at interface 70. The softening and subsequent hardening of material at interface 70 forms a plastic weld 72, shown in the detailed enlargement of FIG. 5, so as to bond together first and second components 62 and 64. It can be seen from the detailed enlargement in FIG. 5 that some mixing of softened, potentially liquefied and flowed, materials at interface 70 has occurred. In certain embodiments, each of components 62 and 64 may be thermoplastic materials such as nylon, such that each material of each component is softened at the interface, and some mixing of the materials at interface 70 occurs prior to hardening and completion of the plastic weld. In the case of certain thermoset materials, some softening might or might not occur in response to the heat, but typically at least one of the components to be bonded together will be thermoplastic or not completely crosslinked, so that softening and then plastic welding occurs upon cooling and hardening when iris 18 is opened to move surfaces 22 out of contact. The present disclosure is nevertheless not limited to plastic welding of any particular materials or combinations of materials. The aforementioned strategies of controlling to a pressure can be used during reducing a diameter of assembly 60 from a first diameter to a second diameter less than the first diameter in response to the compressive pressure, such that application of the compressive pressure is continuous from the state where the assembly has the first diameter to the state where the assembly has the reduced, second diameter.

It will be recalled that conventional tube welding and joining systems utilize jaws having multiple different partial holes within the jaws, differently sized so as to accommodate a plurality of different tube sizes. It is generally necessary to machine the holes, more particularly partial holes in each of two jaws, to relatively exacting tolerances, and then assemble the jaws together also at tight tolerances. A substantial amount of engineering thus goes into designing and implementing conventional tube welding strategies. It is also often necessary to weld tubes together using more than one of the holes in any given jaw assembly. For instance, a technician might commence the plastic welding using a first set of partial holes in the jaws, and then move to a smaller set, to complete the welding process. The present disclosure offers various advantages over such techniques, since the adjustable size iris can effectively clamp and heat the assembly at a range of different diameters, accommodating shrinking of the assembly and continuously applying uniform compressive force in radially inward directions, and substantially uniformly about the assembly.

Referring also to FIG. 6, there is shown a medical device 160 formed from assembly 60, and in particular where device 160 is a balloon catheter with a fitting 76 attached to component 62, and a balloon 74 formed integrally with component 64. Plastic weld 72 bonds together components 62 and 64. Form 68 has been withdrawn from assembly 60 to render the completed medical device 160. Those skilled in the art will envision other medical devices and other locations of plastic welding within medical devices according to the teachings set forth herein.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure.

What is claimed is:

1. A method of making a medical device comprising the steps of:
advancing an assembly of a first component and a second component through an aperture in an iris formed by a plurality of movable contactors in a welding system;
adjusting the iris to narrow the aperture such that heated surfaces of the plurality of movable contactors contact the assembly;
conducting heat from the heated surfaces into the assembly via the contact so as to soften material forming at least one of the first and second components at an interface therebetween; and
forming a plastic weld via the softening and subsequent hardening of the material at the interface so as to bond together the first and second components.

2. The method of claim 1 wherein each of the first and second components has a tubular shape.

3. The method of claim 2 further comprising a step of preventing narrowing of a lumen formed by the first and second components, during the conducting step, via a removable form extending through the lumen.

4. The method of claim 2 wherein the step of adjusting the iris includes adjusting the iris to narrow the aperture about coaxial nested ends of the first and second components.

5. The method of claim 4 wherein the step of adjusting includes adjusting the iris to narrow the aperture such that the heated surfaces contact a shrink tubing material extending about the coaxial nested ends.

6. The method of claim 4 further comprising a step of applying compressive pressure to the assembly via the heated surfaces during the conducting step.

7. The method of claim 6 further comprising a step of reducing a diameter of the assembly from a first diameter to a second diameter less than the first diameter in response to the compressive pressure, and wherein the step of applying further includes continuously applying the compressive pressure during the reducing step.

8. A plastic welding system comprising:
a housing;
a plurality of movable contactors mounted to the housing and forming an iris, the iris defining a center axis and being configured to receive therethrough an assembly of a first component and a second component to be bonded together via plastic welding;
the plurality of movable contactors each further including a surface oriented toward the center axis, such that the surfaces define an aperture of the iris adjustable in size via moving the plurality of movable contactors;
a heating mechanism coupled to the plurality of movable contactors so as to heat the surfaces for conducting heat into the assembly; and
at least one actuator operably coupled to the plurality of movable contactors so as to narrow the aperture to contact the assembly via the surfaces and thereby form a plastic weld bonding together the first and second components.

9. The system of claim 8 wherein the plurality of movable contactors are distributed radially symmetrically about the center axis of the iris and are greater than three in number.

10. The system of claim 9 wherein the plurality of movable contactors are synchronously pivotable about a plurality of pivot axes between first positions where the aperture is larger and the corresponding surface is further from the center axis, and second positions where the aperture is smaller and the corresponding surface is closer to the center axis.

11. The system of claim 10 wherein each of the plurality of movable contactors is in contact with two other ones of the plurality of movable contactors, and slides in contact with the two other ones between the first and second positions.

12. The system of claim 11 wherein each of the plurality of movable contactors has a wedge shape.

13. The system of claim 9 wherein the heating mechanism includes a plurality of resistance heaters each coupled with one of the plurality of movable contactors.

14. The system of claim 8 further comprising a control device in communication with the at least one actuator, and being configured to determine control signals to the at least one actuator so as to controllably apply a compressive pressure to the assembly during the adjustment of the size of the aperture.

15. The system of claim 14 wherein the plurality of movable contactors are adjustable from first positions where the aperture is larger to second positions where the aperture is smaller, and wherein the control device is further configured to controllably apply compressive pressure, via the determined control signals, that is uniform at each of the first and second positions.
